(19) United States
(12) Patent Application Publication
(10) Pub. No.: US 2006/0257490 A1
(43) Pub. Date: Nov. 16, 2006

(54) Formulation based on phospholipids

(76) Inventors: Dirk Cremer, Zolling (DE); Alexander Skolaut, Starnberg (DE); Polona Francic, Freising (DE)

Correspondence Address: FULBRIGHT & JAWORSKI, LLP, 666 FIFTH AVE, NEW YORK, NY, 10103-3198, US

(21) Appl. No.: 10/570,070

(22) PCT Filed: Sep. 3, 2004
(86) PCT No.: PCT/EP04/09861

§ 371 Date: Mar. 1, 2006

(30) Foreign Application Priority Data
Sep. 4, 2003 (DE) 103 40 741.3

(51) Int. Cl. A61K 9/14; A61K 31/715

(52) U.S. Cl. 424/489; 514/54

(57) ABSTRACT

The invention relates to a formulation containing a phospholipid constituent as a physiologically active ingredient, the formulation itself being in a water-dispersible granulated form and containing a fructane constituent, in addition to the phospholipid constituent, as a formulation auxiliary agent, in a weight ratio of 5 to 70: 95 to 30. At least one representative of the series phosphatidylcholine, phosphatidylethanolamine, phosphatidylinosite, phosphatidylserine and/or the lysoforms thereof is used in a preferred formulation. Typical representatives of the fructane constituent are graminan, inulin and levan. Said formulations have a particle size of between 10 and 1 mm, can optionally contain other physiologically active ingredients, and are used as/in food supplements, special foods or functional foods.

The present invention relates to a formulation containing a phospholipid component as physiologically active ingredient, a method for its production, and use thereof.

The substance class of phospholipids is what are termed complex lipids having amphiphilic properties, that is simultaneously lipophilic and hydrophilic properties, which enables them, inter alia, to form lipid double layers in aqueous media. Phospholipids (also termed phosphatides), considered chemically, are phosphodiesters in which the phosphoric acid is esterified firstly with a sphingosine or glyceride radical and secondly with choline, ethanol, amine, serine, inositol or glycerol. Phosphatidylcholine is also known as lecithin and at the same time gives the name to a large group of specific phospholipids, namely the lecithins. Phosphatidylserine and phosphatidylethanolamine are also termed cephalins.

The lyso derivatives also belonging to this group are formed by hydrolytic cleavage in the C₁ and/or C₂ position of the glycerol moiety using specific phospholipases.

On account of their amphiphilic properties, phospholipids are also used as sheath substances of the known liposomes and transferosomes. In this context, they are used especially on account of their bioadhesive properties, in the sector of mucosal tissue applications, with them being introduced in particular into the nasal and oral cavities.

In chemically modified form, phospholipids, however, are also used as surface-active formulation aids (surfactants). It is also known to produce vesicles by using sonication, which carry phospholipids as sheath. From the prior art, capsules are also adequately known which likewise carry phospholipids as sheath substance. If phospholipids are used in the filling, that is in the capsule core, they act there usually in small amounts as formulation aids having usually solubilizing properties.

Special granules having lecithin sheaths are disclosed by Japanese application JP 9147043, and also by EP-A 493441. These granulates which contain, inter alia, steroids as bioactive substances, are used as feed additives.

According to WO 87/04347, lysophospholipids are described as solubilizers for hydrophobic bioactive substances.

Phospholipids in liquid form have been previously described in connection with hard or soft gelatin capsules; however, formulations of phosphatidylserine and phosphatidylcholine, in particular in a mixture with other lecithins and/or oils, in soft capsules have proved to be insufficiently stable.

A consequence of this described instability of lecithins as capsule contents is that, instead of a desired liquid formulation, at best moderately pasty blends can be encapsulated.

Difficulties with respect to the processibility and stability of phospholipid formulations exist, however, not only in connection with predominantly lipophilic administration forms, but especially also in the case of formulations in powder form, and here in particular with respect to their water dispersibility.

For this reason many attempts have been made to produce improved phospholipid formulations in powder form, attention principally being directed to water-dispersible forms.

For instance, Japanese patent JP 63185929 discloses the production of a dispersible lecithin powder having a high phosphatidylcholine content in which a dispersion of the lecithin in a fat or oil is first mixed with an aqueous solution and/or dispersion of a water-soluble protein or cellulose powder and subsequently the resultant blend is spray dried. The water-soluble protein and/or the cellulose powder acts here as sheath material and is used in the form of an aqueous dispersion. This method produces a ready-to-use composition having a high phosphatidylcholine content and good dispersion properties in water, so that it is suitable especially for producing aqueous dispersions of low viscosity.

According to JP 11289993, a water-dispersible lecithin composition is obtained by introducing into water lecithin having a high degree of purity together with ethanol and a water-soluble polymer. Suitable water-soluble polymers for this are, in particular, sodium caseinate, carrageenans, xanthan gum, hydroxypropyl-cellulose and modified starch. The lecithin composition obtained in this manner can be used to develop a stable emulsion having a low viscosity and very good handling ability.

Production of a solid lecithin or lecithin in powder form is described in JP 2779236. According to this, first a dispersion containing lecithin, polyalcohol and a hydrophilic polyoxyethylenealkyl ester or ether is charged, subsequently dehydrogenated and finally dried. The lecithin component used can preferably comprise an electrolyte such as ascorbic acid, pyrrolidone carboxylate, amino acids, citric acid and suitable salts thereof.

A mixture containing lecithin and a styrene component in portions <5% by weight is described in JP 62126966. Adding lecithin and a styrene component to an instant powder, such as milk powder, dry soups, cocoa and coffee powder and also cream powder, significantly increases its solubility or dispersibility in cold water.

Overall, it is still disadvantageous with all these described methods, in particular with respect to achieving a water-dispersible phospholipid powder, that by using the phospholipid-containing products thus obtainable, only more or less water-dispersible powders are obtained. Thus, on water contact, agglomerate formation still occurs which is caused by the hydrophilic-hydrophobic interaction between the phospholipid component and the surrounding water-containing medium. Even spray-drying methods or so called “top spray”-methods and/or adding solubilizers such as maltodextrin and fat caseinate and/or alcoholic components do not lead to a rapid and silmultaneously improved water dispersibility without agglomerate formation.

On account of the described disadvantage of the prior art, for the present invention the object is set of providing a formulation which, as physiologically active ingredient, contains a phospholipid component and which has significantly improved properties with respect to its water dispersibility.

This object was achieved using a corresponding formulation which is present in water-dispersible granulate form and which, in addition to the phospholipid component, contains a fructan component as formulation aid in the weight ratio 5 to 70:95 to 30.

Fructans are compounds having at least one fructosyl-fructose bond, for example as present in inulin. They are therefore fructooligosaccharides, or simply “oligofructoses”, which occur as widely distributed carbohydrates in nature. They are found principally in over 30 000 plant families and are also used as physiologically active components in human nutrition. High fractions of fructan and, in particular, inulin, as natural ingredients are found, in particular, in plant fibers, but also in storage organs of plants, increased fructan fractions in concentrations of 0.3 to 6% by weight being found especially in wheat, onions, bananas, chicory, leek and sugar beets.

Inulin has a degree of polymerization of 50 to 60 fructose units and is produced technologically from chicory roots.

The β-(b 2-1)-bonds of the fructose units which are typical of fructans cannot be cleaved by enzymes in the gastrointestinal tract; inulin and other oligofructoses, however, are partially fermented in the large intestine, for example by bifidobacteria, which is why fructans are also described as what are termed soluble food fibers (dietary fibers) having extremely low energy value.

Inulin, in addition, is distinguished in that, in water, it is able to form microcrystals, which cause a creamy taste. Inulin is therefore also a low-calorie fat substituent. Oligofructoses display similar characteristics as glucose syrup and are thus, because of their sweet taste, which reaches roughly a third of the intensity of sucrose, used as sugar substituent in light products, but also as moisture-retention agent in bakery products.

Surprisingly, it has proved with the inventive formulation that phospholipids, and in particular lecithins having a high phosphatidylserine portion, together with fructans, for example inulin, with simultaneous use of deionized water, can be converted under simple granulation to give a highly water-dispersible formulation. On account of the gentle processing, scarcely any losses occur in the main ingredients and, in addition, an in part complex industrial post treatment, for example in the form of spray drying, with high energy input, can be omitted. Also, the fructans used for the formulation are aids suitable for foodstuffs which are readily available in relatively large amounts, for which reason the achievable formulations can be prepared without great technical expense in an economically favorable manner.

These advantages were not predictable to this extent.

With respect to the phospholipid component used, the present inventive formulation is not subject to any restriction. However, phospholipid mixtures, such as lecithins, for example, but in particular defined phospholipids, such as phosphatidylcholine, phosphatidylethanolamine, phosphatidylinositol, phosphatidylserine and/or lyso forms thereof have been found to be particularly suitable.

With respect to the phospholipid portion of the total formulation, minimum portions of 2.0% by weight are to be preferred just as the minimum portions of 5.0% by weight of the particularly suitable phosphatidylserine.

With respect to the lecithins which are highly suitable for the formulation, varieties have proved to be expedient which have a phosphatidylserine content of at least 20% by weight, wherein phosphatidylserine contents of more than 30, and in particular more than 45% by weight, are likewise highly suitable and, obviously, wherein the minimum portions in the total formulation being taken into account.

The wide range of use of the formulation claimed by the present invention is also made clear by the free choice of fructan component. This is because it is not restricted to a certain representative, so that graminan, inulin and levan can be used equally well as formulation aids in the context of the invention. Obviously, here, in addition to the pure compounds, it is also possible to use any desired mixtures.

The advantageous width of the claimed formulation is clear not only due to the high variability with respect to the main components, but also in the suitable mixing ratio between phospholipid component and the fructan component. For instance, preferably ratios between the phospholipid component and the fructan component of 10 to 40:90 to 60, and particularly preferably 15 to 30:85 to 70, are possible.

Although the pronounced water dispersibility of the claimed granulates is principally due to the formulation aid used, depending on the respective field of use, the water dispersibility can also be improved by a residue water content. For this reason, the present invention also takes into account a special formulation, the water content of which ranges from 0.01 to 10% by weight. This water content, which customarily makes up 0% by weight, affects not only the water dispersibility of the total formulation, but it also makes it possible to affect the physical properties of the granulates.

In this context, the present invention also considers formulations as particularly preferred whose granulates have a particle size from 10 μm to 1 mm, and particularly preferably from 100 to 300 μm, the main fraction being in the range from 125 to 200 μm.

In addition to the main components, that is to say the phospholipid component and the fructan component, the claimed formulation, however, can also contain further physiologically active components and/or formulation aids, such as, e.g. carbohydrates, vitamins, alcohols, amino acids, fatty acids, carboxylic acids, salts, fibers, flavorings and colorings of natural or synthetic origin, which are different from the main components.

Supplementary to the claimed formulation, the present invention also comprise a method for producing this formulation, wherein the phospholipid component is charged in a granulating drum with the fructan component in the weight ratio 5 to 70:95 to 30, if appropriate with further physiologically active components, subsequently admixed with 5 to 20% by weight, based on the dry mixture, at least once-deionized water and subsequently granulated to a particle size of from 10 μm to 1 mm. This method for which, in particular, a twice-deionized water should be used, is preferably carried out until the water content of the granules is 0.01 to 10% by weight, a water content of 0.01% by weight being general.

The advantages of the claimed formulation are exhibited not only in its relatively simple structure and the overall simple economic ease of production, but also in the broad possibilities of application. In this respect, the invention comprises the use of the claimed formulation as food supplement and special diet, but also for the production of food supplements, special diet and functional foods. Obviously, the claimed formulations, however, can also be used in cosmetics and as feed additives.

With respect to the broadness of the possible fields of application, for the inventive formulations, applications for increasing mental and physical performance, in distress and also sport-related physical stress, and also in attention-deficit hyperactivity disorder (ADHD), are in the foreground.

Overall, the present invention comprises a formulation which is available in an extremely economic manner in a short time with low use of production aids and which, as a finished product, compared with the prior art, is outstandingly suitable for preparing aqueous dispersions, it forming no agglomerates on contact with the aqueous medium, and, in powder form, dispersing and distributing immediately in water. On dispersion with particle sizes in the lower diameter range, in this manner virtually clear, aqueous dispersions are obtained without perceptible particles, which is beneficial, in particular with respect to consumer acceptance, especially when the claimed formulations, as also proposed, are used as food supplements or functional food, for example in aqueous drinks.

The advantages described are shown by the example hereinafter.

### EXAMPLE

200 g of a phospholipid component (Leci PS® 20 P from Degussa Food Ingredients GmbH) were charged with 800 g of a fructan component (Raftilose® from Orafti NA) in a granulation drum and are then sprayed during the granulation operation with 80 g of a twice-deionized water at room temperature.

The granulation operation was completed after 12 minutes. Possible under particle size or over particle size fractions were not separated off from the resultant raw particle. The resultant finished particle having a ratio of phospholipid component:fructan component of 1:4 had the following particle size distribution:
63 to 125 μm: 22%
125 to 200 μm: 51%
200 to 250 μm: 16%
250 to 500 μm: 10%
>1 mm: <0.2%

The residue water content was 0.01% by weight. This formulation can readily be dispersed in cold and also warm water, and without any agglomerate formation and has pronounced long-term stability with respect to the granules, and without showing any settling behavior.

1-13. (canceled)

14. A formulation comprising (a) a phospholipid component wherein said phospholipid component is present in water-dispersible granulate form and (b) a fructan component as a formulation aid in the weight ratio 5 to 70:95 to 30.

15. The formulation of claim 14, wherein said phospholipid component comprises at least one compound selected from the group consisting of: phosphatidylcholine, phosphatidylethanolamine, phosphatidylinositol, phosphatidylserine or lyso forms thereof.

16. The formulation of claim 14, wherein said phospholipid component of the total formulation is at least 2.0% by weight.

17. The formulation of claim 14, wherein said phospholipid component is phosphatidylserine and comprises at least 5.0% by weight of the total formulation.

18. The formulation of claim 14, wherein said phospholipid component is a lecithin, having a phosphatidylserine content of at least 20% by weight.

19. The formulation of claim 14, wherein said fructan component comprises at least one compound selected from the group consisting of graminan, inulin and levan.

20. The formulation of claim 14, wherein the weight ratio of phospholipid component to fructan component is 10 to 40:90 to 60.

21. The formulation of claim 14, wherein the weight ratio of phospholipid component to fructan component is 15 to 30:85 to 70.

22. The formulation of claim 14, wherein said formulation has a water content of 0.01 to 10% by weight.

23. The formulation of claim 14, wherein the granulates have a particle size from 10 μm to 1 mm.

24. The formulation of claim 14, wherein the granulates have a particle size from 1100 to 300 μm.

25. The formulation of claim 14, further comprising a physiologically active component or a formulation aid selected from the group consisting of a carboxylic acid, a salt, a fiber, a flavoring and a coloring.

26. A method for producing a formulation as claimed in claim 14, comprising a) charging the phospholipid component in a granulating drum together with the fructan component in the weight ratio 5 to 40:95 to 60, b) subsequently admixing with 5 to 20% by weight, based on the dry mixture, of at least once-deionized water and c) subsequently granulating to a particle size of from 10 μm to 1 mm.

27. The method of claim 26, wherein the granulation is carried out until the water content of the granulates is 0.01 to 10% by weight.

28. A food supplement, diet supplement or functional food comprising the formulation of claim 14.

29. The formulation of claim 25, wherein the physiologically active component or formulation aid is natural or synthetic.
